(19) United States
(12) Patent Application Publication
(10) Pub. No.: US 2001/0046502 A1
(43) Pub. Date: Nov. 29, 2001

(54) Animal model for testing immunotherapies of spontaneous metastatic disease

(75) Inventor: Margalit B. Mokyr, Chicago, IL (US)

Correspondence Address: DARBY & DARBY P.C., 805 Third Avenue, New York, NY, 10022, US

(73) Assignee: The Board of trustees of the University of Illinois

(21) Appl. No.: 09/775,537

(22) Filed: Feb. 1, 2001

Related U.S. Application Data
(60) Provisional application No. 60/180,257, filed on Feb. 4, 2000.

(51) Int. Cl. A61K 49/00; A01K 67/00; A61K 39/00

(52) U.S. Cl. 424/277.1; 424/9.2; 800/10

(57) ABSTRACT

The present invention provides an animal model that can provide information on the effectiveness of postsurgical immunotherapies for recurrence of metastatic disease. In a specific embodiment, this tumor model used mice from which the primary 410.4 mammary carcinoma was surgically excised to assess the therapeutic potential of low-dose cyclophosphamide (CY) followed by vaccination with DNP-modified, γ-irradiated, autologous tumor cells admixed with BCG compared to mice receiving low-dose CY followed by vaccination with unmodified, γ-irradiated, autologous tumor cells admixed with BCG, or mice treated with PBS (control group). In this model, therapeutic benefits offered by DNP-modified, γ-irradiated, autologous tumor cell vaccine (preceded by low-dose CY) were abrogated completely upon depletion of CD8⁺ T-cells, and was improved when the mice were pretreated with a single dose of DNP-modified, γ-irradiated, autologous tumor cells (without BCG) prior to the low-dose CY treatment, and then subjected to vaccination with DNP-modified, γ-irradiated, autologous tumor cells admixed with BCG.

### FIELD OF THE INVENTION

The present invention relates to an animal model that provides information about the efficacy of an immunotherapy for metastatic disease more accurately than prior animal models. More specifically, the invention permits evaluation of haptenized tumor cell vaccines.

### BACKGROUND OF THE INVENTION

### Haptenized Tumor Cell Vaccines

An autologous whole-cell vaccine modified with the hapten dinitrophenyl (DNP) has been shown to produce inflammatory responses in metastatic sites of melanoma patients. The survival rates of patients receiving post-surgical adjuvant therapy with DNP-modified vaccine are markedly higher than those reported for patients treated with surgery alone. Intact cells are preferred for the vaccine.

U.S. Pat. No. 5,290,551, to David Berd, discloses and claims vaccine compositions comprising haptenized melanoma cells. Melanoma patients who were treated with these cells developed a strong immune response. This response was detected, e.g., in a delayed-type hypersensitivity (DTH) response to haptenized and non-haptenized tumor cells. More importantly, the immune response to non-haptenized cells has been associated with an increased survival rate of melanoma patients.

Haptenized tumor cell vaccines have also been described for other types of cancers, including lung cancer, breast cancer, colon cancer, pancreatic cancer, ovarian cancer, and leukemia (see U.S. patent application Ser. No. 08/203,004, filed Feb. 28, 1994; PCT Publication Nos. WO96/40173 and WO98/14026, and PCT Application No. PCT/US98/16660).

Generally, the immune response to haptenized cells has been found to be independent of the choice of hapten, but dependent on the functional group to which the hapten is attached. In particular, it has been reported that haptenization of ε-amino groups of lysine and —COOH groups of aspartic acid and glutamic acid is effective for a robust immune response (Nahas and Leskowitz, Cellular Immunol., 1980;54:241).

These discoveries have led to rapid advances in the treatment of cancer, particularly melanoma, by immunotherapy. Nevertheless, there remains a need in the art for even more effective therapies, since the response rates achieved with the haptenized tumor cell vaccine technologies mentioned above, while impressive, have not reached 100%. There is also a need in the art for more effective treatment regimens which require substantially fewer number haptenized cells per dose, either to permit more dosages or to provide an effective therapy with a smaller number of cells. This is especially critical for the treatment of an early stage or recurrent cancer, when the number of cells obtainable from a resected tumor may be fewer than necessary for vaccine preparation as described above. To make further advances as rapidly as possible requires a suitable animal model.

### Animal Models of Tumor Immunotherapy

Tumors are typically generated in animals by induction, e.g., using a carcinogen such as a petroleum oil, in immunocompromised animals with a tumor xenograft, or by administration of cells of a syngeneic tumor cell line.

Although immunotherapy is actively being tested in animal models against primary tumors and experimental metastases, very few studies have examined the effectiveness of immunotherapy against spontaneously occurring metastatic disease. However, the latter type of animal tumor models is likely to be more relevant for the assessment of the potential of an immunotherapeutic protocol for the treatment of cancer patients because in cancer patients the primary tumors are surgically removed before the patients are subjected to immunotherapy in an attempt to eradicate or slow down the progression of metastases.

It is known from animal studies that immunization of mice with syngeneic lymphocytes modified with arsanilic acid induces strong T cell responses against those modified cells, including DTH (Bach et al., J. Immunol., 1978;121:1460) and cytotoxic T cells (Sherman et al., J. Immunol., 1978; 121:1432). Injection of arsanilic acid into the rat kidney induced a brisk autoimmune nephritis (Rennke et al., Kidney International, 1994;45:1044). Obviously, the administration of even minute amounts of arsanilic acid into human is unacceptable, but sulfanilic acid, a non-toxic compound in small amounts, should induce a similar immunological effect (Nahas and Leskowitz, supra, 1980). Both compounds can be coupled to tyrosine and histidine after being diazotized by treatment with sodium nitrite. Moreover, immunization of animals with sulfanilic acid-modified protein can induce autoimmunity (Weigle, J. Exp. Med., 1965; 122:1049). A third potentially interesting hapten in this category is phosphorylcholine (PC), in light of the work of Kim et al. (Eur. J. Immunol., 1992;22:775). However, it has not been established that these haptens will be effective in humans; on the contrary, Nahas and Leskowitz, supra, suggest otherwise. Nor have the animal models been related to treatment of human cancer.

Berd and his associates provided data consistent with a role for CD8⁺ T-cells in the anti-metastatic effects of this regimen of DNP-modified, γ-irradiated, autologous tumor cell vaccine. Specifically, they have shown that DNP-modified, γ-irradiated, autologous tumor cell vaccine induced a striking inflammatory response in superficial metastases which consisted predominately of CD8⁺ T-cells (Berd et al., Cancer Res 1991;51:2731; Sato, Cancer Immunol Immunotherapy 1996;43:174). Moreover, analysis of T cell receptor (TCR) Vβ usage by these infiltrating CD8⁺ T-cells illustrated preferential utilization of a particular Vβ gene segment, the Vβ14 (Sato, Cancer Immunol Immunotherapy 1996;43:174; Sensi et al., J Clin Invest 1997;99:710), suggesting selective conal expansion of these CD8⁺ T-cells in response to DNP-modified, γ-irradiated, autologous tumor cell vaccine. However, to date there are no data for the consequences of immunotherapy in a CD8⁺-deficient or Vβ repetoire-deficient, system. A suitable animal model would permit such investigations to proceed.

The present invention addresses these and other needs in the art in a surprisingly effective way.

The citation of any reference herein should not be construed as an admission that such reference is available as “Prior Art” to the instant application.

### SUMMARY OF THE INVENTION

The present invention advantageously provides a method for studying cancer treatment in an animal model. The method is particularly useful for the in vivo evaluation of compositions comprising haptenized tumor cells or tumor cell extracts intended to induce an anti-tumor response in a cancer patient.

In one embodiment, the method enables testing the efficacy of an immunotherapy of a tumor in a non-human animal. Preferably, the method comprises evaluating an anti-tumor response to a tumor immunotherapy administered to a non-human animal harboring a tumor. The tumor is preferably metastatic, and preferably spontaneous.

In another embodiment, the method enables testing the efficacy of an immunotherapy of a spontaneous carcinoma tumor in a mouse. Preferably, the method comprises evaluating an anti-tumor response to a carcinoma immunotherapy given to a mouse harboring a spontaneous carcinoma tumor, in which the immunotherapy comprises administering a composition comprising a hapten-modified carcinoma tumor cell preparation to the mouse.

### DESCRIPTION OF THE DRAWINGS

FIG. 1. Therapeutic effectiveness of DNP-modified, γ-irradiated, autologous tumor cell vaccine against metastatic disease in the murine 410.4 tumor model. Relapse-free survival of mice in which the primary 410.4 tumor was excised when it reached 6-8 mm in diameter and the mice were subjected to low-dose CY followed by DNP-modified γ-irradiated, autologous tumor cell vaccine (▪ [solid square]) or low-dose CY followed by unmodified, γ-irradiated, autologous tumor cell vaccine (□ [open square]). As reference, we provide the relapse-free survival of mice treated with PBS only (control) (▴ [solid triangle]). The number of mice in each group is provided in parenthesis next to the symbol for this group in the legend located in the body of the figure.

FIG. 2. Importance of CD8⁺ T-cells for the therapeutic effectiveness of DNP-modified, γ-irradiated, autologous tumor cell vaccine against metastatic disease in the murine 410.4 tumor model. Relapse-free survival of mice depleted of CD8⁺ T-cells by treatment with anti-CD8 mAb (∘ [open circle]) or of mice treated with normal IgG (▪ [solid square]) and subjected to low-dose CY treatment followed by DNP-modified γ-irradiated, autologous tumor cell vaccine. As reference, we provide the relapse-free survival of mice depleted of CD8⁺ T-cells (● [solid circle]), or of mice treated with normal IgG (□ [open square]) and subjected to low-dose CY treatment followed by unmodified γ-irradiated, autologous tumor cell vaccine. The number of mice in each group is provided in parenthesis next to the symbol for this group in the legend located in the body of the figure.

FIG. 3. Potentiating effects of pretreatment with DNP-modified, γ-irradiated, autologous tumor cells for the anti-metastatic activity of low-dose CY followed by DNP-modified, γ-irradiated, autologous tumor cell vaccine in the murine 410.4 tumor model. Relapse-free survival of mice that were (▴ [solid triangle) or were not (▪ [solid square]) pretreated with DNP-modified γ-irradiated, autologous tumor cells 3-7 days prior to initiation of the low-dose CY treatment followed by DNP-modified γ-irradiated, autologous tumor cell vaccine. As reference, we provide the relapse-free survival of mice subjected to low-dose CY followed by unmodified γ-irradiated, autologous tumor cell vaccine (□ [open square]). The number of mice in each group is provided in parenthesis next to the symbol for this group in the legend located in the body of the figure.

### DETAILED DESCRIPTION OF THE INVENTION

The present invention is based, in part, on work with an animal model that can provide information on the effectiveness of postsurgical immunotherapies for recurrence of metastatic disease. Utilizing this tumor model, which consists of mice from which the primary 410.4 mammary carcinoma was surgically excised, the therapeutic potential of low-dose cyclophosphamide (CY) followed by vaccination with DNP-modified, γ-irradiated, autologous tumor cells admixed with BCG was assessed. Example 1, infra, revealed that low-dose CY treatment followed by vaccination with DNP-modified, γ-irradiated, autologous tumor cells admixed with BCG improved the relapse-free survival of mice as compared to mice receiving low-dose CY followed by vaccination with unmodified, γ-irradiated, autologous tumor cells admixed with BCG, or mice treated with PBS (control group).

Example 2 further shows that therapeutic benefits offered by DNP-modified, γ-irradiated, autologous tumor cell vaccine (preceded by low-dose CY) were abrogated completely upon depletion of CD8⁺ T-cells, illustrating the importance of CD8⁺ T-cells for the anti-metastatic effects of this immunotherapeutic protocol.

In Example 3, improvement in the therapeutic outcome of DNP-modified, γ-irradiated, autologous tumor cell vaccine preceded by low-dose CY was observed when the mice were pretreated with a single dose of DNP-modified, γ-irradiated, autologous tumor cells (without BCG) prior to the low-dose CY treatment, and then subjected to vaccination with DNP-modified, γ-irradiated, autologous tumor cells admixed with BCG. These results illustrate the potentiating effect of the pretreatment regimen for the vaccination protocol.

The various aspects of the invention will be set forth in greater detail in the following sections. This organization into various sections is intended to facilitate understanding the invention, and is in no way intended to be limiting thereof.

### Definitions

The following defined terms are used throughout the present specification, and should be helpful in understanding the scope and practice of the present invention.

In a specific embodiment, the term “about” or “approximately” means within 50%, preferably within 25%, and more preferably within 10% of a given value or range. Alternatively, the term about means within an acceptable standard error of the mean, when considered by one of ordinary skill in the art.

A non-human animal is any animal, e.g., mammal or avian species, and preferably a mammal, that can serve as an animal model for testing anti-tumor immunotherapies. Preferred animals are laboratory animals, preferably rodents, e.g., mice, rats, hamsters, guinea pigs and the like. Other suitable laboratory animals are of course rabbits, cats, dogs, small monkeys, and apes. In addition, certain farm animals are also often employed as laboratory animals, notably chickens, goats, sheep, and pigs. Preferably these animals readily develop or can be transplanted with an autologous (syngeneic) spontaneous tumor. In a specific embodiment, the laboratory animal is a mouse; more specifically, a BALB/c mouse. Non-human animals are readily available from researchers or commercial suppliers, such as Jackson Laboratories (Bar Harbor, Me.), Charles River Breeding Laboratories (Wilmington, Mass.), Taconic Farms (Germantown, N.Y.), to mention a few such suppliers.

The phrase “pharmaceutically acceptable” refers to molecular entities, at particular concentrations, and compositions that are physiologically tolerable and do not typically produce an allergic or similar untoward reaction, such as gastric upset, fever, dizziness and the like, when administered to a human or non-human animal. Preferably, as used herein, the term “pharmaceutically acceptable” means approved by a regulatory agency of the Federal or a state government or listed in the U.S. Pharmacopoeia or other generally recognized pharmacopoeia for use in humans or non-human animals.

As used herein, the term “isolated” means that the referenced material is removed from the natural environment in which it is normally found. In particular, isolated biological material if free of cellular components. An isolated peptide may be associated with other proteins or nucleic acids, or both, with which it associates in the cell, or with cellular membranes if it is membrane-associated. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably free of other proteins or nucleic acids with which it is associated in a cell; a purified cell is free of unrelated cells and tissue matrix components.

The term “free of any adjuvant” means that the composition of a tumor cell preparation does not contain adjuvant and is not administered with an adjuvant, nor is adjuvant given less than 24 hours before or after the composition. This is also referred to as “adjuvant free.”

A “subject” is a non-human animal who may receive haptenized tumor cells formulated in a composition of the invention.

An “anti-tumor response” is at least one of the following: tumor necrosis, tumor regression, tumor inflammation, tumor infiltration by activated T lymphocytes, activation of tumor infiltrating lymphocytes, delayed-type hypersensitivity (DTH) response, or a clinical response. Clinical response criteria for anti-tumor response resulting from treatment according to the present invention include complete, partial, or mixed response, as well as stable disease. Other clinical responses that may be observed upon following the treatment of the invention is prolongation of time to relapse, or prolongation of survival.

A “formulation” refers to an aqueous medium or solution for the preservation or administration, or both, of haptenized tumor cells or tumor cell extracts, which is preferably directly injectable into an organism. The aqueous medium will include salts or sugars, or both, at about an isotonic concentration.

A “composition” is an admixture of a tumor cell preparation (preferably haptenized) in a formulation.

A “vaccine composition” is a composition as set forth previously further comprising an adjuvant, including an immunostimulatory cytokine or lymphokine.

The terms “vaccine”, “immune therapy” and “immunotherapy” are used herein interchangeably to administration of a composition comprising a tumor cell preparation (preferably haptenized) to treat a cancer, e.g., after surgical resection of the tumor. “Efficacy of an immunotherapy” is the degree to which the immunotherapy elicits am anti-tumor response in an individual subject, or the percentage of subjects in which an anti-tumor response develops as a result of treatment. Preferably efficacy is determined by composition to controls that harbor the spontaneous tumor but receive either no therapy, sham therapy, or an alternative therapy.

A “tumor cell preparation” refers to isolated or purified tumor cells or a tumor cell extract for inclusion in a composition. “Hapten modified” means that the tumor cells (or extract) are chemically coupled (conjugated) to a hapten, as that term is understood immunology.

The term “treat” means to attempt to elicit an anti-tumor response against cells of the tumor, i.e., the cancer.

### Haptenized Tumor Cell Preparation

The present invention is directed for use in evaluating haptenized tumor cell vaccines for treating cancer, including metastatic and primary cancers, in humans and non-human animals. Cancers treatable with the present invention include solid tumors and non-solid tumors, including hematologic malignancies. Examples of solid tumors that can be treated according to the invention include sarcomas, carcinomas, and other tumors such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendothelio-sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Hematologic malignancies include leukemias, lymphomas, and multiple myelomas. The following are non-limiting preferred examples of the cancers treatable with the composition and methods of the present invention: melanoma, including stage-4 melanoma; ovarian, including advanced ovarian; leukemia, including but not limited to acute myelogenous leukemia; colon, including colon metastasized to liver; rectal, colorectal, breast, lung, kidney, and prostate cancers.

These same spontaneous tumors from non-human animals can serve as spontaneous tumors in the practice of this invention. As used herein, the term “spontaneous tumor” refers to a tumor that arises (or arose in the past) naturally, in contrast to tumors elicited by treating the subject with as carcinogen, or by transplantation with an allogeneic or xenogenic tumor cell line. A spontaneous tumor can be a maintained as a tumor cell line an implanted into an animal. Tumor cells or cell lines derived from spontaneous tumors may be genetically manipulated, e.g., to express a marker gene or some other gene of interest. Examples of such cell lines include the B16 melanoma of C517BL/6 (mouse) origin; the 410.4 mammary carcinogen of BALB/c origin; the PPC-5 plasmacytoma of BALB/C origin; and the P815 mastocytoma of DBA/2 (mouse) origin. These cell lines can be obtained from various sources, such as the American Type Culture Collection (ATCC) (Manassas, Va.). For example, B16 cells (accession nos. CRL-6322, CRL-6323, and CRL-6475) and P815 cells (TIB-64) are available from the ATCC (http://www.atcc.org). Preferably, tumor cells from a spontaneous tumor cell or cell line express low levels of MHC class I molecules and are weakly immunogenic.

### Tumor Cells

The compositions of the present invention are prepared from tumor cells, e.g, cells obtained from tumors surgically resected as described above by isolating the cells from resected tumors or from in vitro cultures. Tumor cells to be used in the present invention may be prepared as described previously for treating human tumors. Tumors can be processed as described by Berd et al., Cancer Res., 1986;46:2572, Sato, et al., Cancer Invest., 1997;15:98, U.S. Pat. No. 5,290,551, and applications U.S. Ser. Nos. 08/203,004, 08/479,016, 08/899,905, 08/942,794, or corresponding PCT Publication WO 96/40173, each of which is incorporated herein by reference in its entirety. Briefly, the cells are extracted by dissociation, such as by enzymatic dissociation with collagenase and DNase, by mechanical dissociation in a blender, by teasing with tweezers, using mortar and pestle, cutting into small pieces using a scalpel blade, and the like. With respect to liquid tumors, blood or bone marrow samples may be collected and tumor cells isolated by density gradient centrifugation.

The tumor cells of the present invention may be intact, attenuated, or killed cells. Tumor cells incapable of growth and division after administration into the subject, such that they are substantially in a state of no growth, are preferred for use in the present invention. It is to be understood that “cells in a state of no growth” means intact cells that will not divide.

Conventional methods of rendering cells incapable of division are known to skilled artisans and may be useful in the present invention. For example, cells may be irradiated prior to use. Tumor cells may be irradiated to receive a dose of about 2500 cGy to prevent the cells from multiplying after administration. Alternatively, haptenization, and particularly dual haptenization, can render the cells incapable of growth.

The tumor cells should preferably originate from the same type of cancer as that to be treated, and are even more preferably syngeneic (e.g., autologous or tissue-type matched). For purposes of the present invention, syngeneic refers to tumor cells that are closely enough related genetically that the immune system of the intended recipient will recognize the cells as “self”, e.g., the cells express the same or almost the same complement of MHC molecules.

Another term for this is “tissue-type matched.” For example, genetic identity may be determined with respect to antigens or immunological reactions, and any other methods known in the art. A syngeneic tumor cell can be created by genetically engineering a tumor cell to express the required MHC molecules.

Preferably the cells originate from the type of cancer which is to be treated. The tumor cells may be, but are not limited to, autologous cells dissociated from biopsy or surgical resection specimens, or from tissue culture of such cells.

### Tumor Cell Membranes

The isolated, modified tumor cell membranes of the present invention are prepared from tumor cells as set forth above. In one embodiment of the invention, tumor cell membranes are isolated from a spontaneous tumor of an animal, e.g., from a rodent, feline, canine, equine, bovine, or porcine family. Isolation and preparation of haptenized tumor cell membranes is described in U.S. patent application Ser. No. 08/479,016, filed Jun. 7, 1995 and U.S. application Ser. No. 90/025,012, filed Feb. 17, 1998. Preferably, in such a case, the tumor cell membrane preparation is not contaminated with tumor cells capable of multiplying in vivo.

As with tumor cells, tumor cell membranes are preferably isolated from the tumor cells of the same type of cancer as that to be treated. For example, membranes to be used for treating ovarian cancer are isolated from ovarian cancer cells. Preferably, the tumor cells originate from the same subject who is to be treated. The tumor cells are preferably syngeneic (e.g. autologous), but may also be allogeneic to that subject. There may be genetic identity between a particular antigen on the tumor cell used as a membrane source and an antigen present on the patient's tumor cells. The tumor cells may be, but are not limited to, cells dissociated from biopsy specimens or from tissue culture. Membranes isolated from allogeneic cells and stem cells are also within the scope of the present invention.

Tumor cell membranes may include all cellular membranes, such as outer membrane, nuclear membranes, mitochondrial membranes, vacuole membranes, endoplasmic reticular membranes, golgi complex membranes, and lysosome membranes. In one embodiment of the invention, more than about 50% of the membranes are tumor cell outer membranes. Preferably, more than about 60% of the membranes consist of tumor cell outer membranes, with more than about 70% being more preferred, 80% being even more preferred, 90% being even more preferred, 95% being even more preferred, and 99% being most preferred.

Preferably, the isolated membranes are substantially free of nuclei and intact cells. For example, a membrane preparation is substantially free of nuclei or intact cells if it contains less than about 100 cells and/or nuclei in about 2×10⁸ cell equivalents (c.e.) of membrane material. A cell equivalent is that amount of membrane isolated from the indicated number of cells. An isolated tumor cell membrane which is substantially free of cells and/or nuclei may contain lymphocytes and/or lymphocyte membranes.

Preferably, the isolated tumor cell membranes are the outer cell membranes, i.e., tumor cell plasma membranes. The membrane preparation of the invention may contain the entire outer membrane or a fraction thereof. An isolated membrane of the invention, preferably including a fraction of the outer membrane, contains an MHC molecule fraction and/or a heat shock protein fraction. The size of the membrane fragments is not critical.

Allogeneic tumor cell membranes may also be used in the methods of the present invention with syngeneic (e.g. autologous) antigen presenting cells. This approach permits immunization of a subject with tumor cell membranes originating from a source other than the patient's own tumor. Syngeneic antigen-presenting cells process allogeneic membranes such that the patient's cell-mediated immune system may respond to them.

A tumor cell membrane (modified or un-modified) as referred to in this specification includes any form in which such a membrane preparation may be stored or administered, such as, for example, a membrane resuspended in a diluent, a membrane pellet, or a frozen or a lyophilized membrane.

The tumor cell membranes can be obtained from haptenized cells, or may be haptenized after extraction from the cells using the techniques described infra.

Tumor cell membranes are prepared from tumor cells, e.g., obtained as described above, by disrupting the cells using, for example, hypotonic shock, mechanical dissociation and enzymatic dissociation, and separating various cell components by centrifugation. Briefly, the following steps may be used: lysing tumor cells, removing nuclei from the lysed tumor cells to obtain nuclei-free tumor cells, obtaining substantially pure membranes free from cells and nuclei, and coupling the tumor cell membranes to a hapten to obtain hapten-modified tumor cell membranes. Membrane isolation may be conducted in accordance with the methods of Heike et al.

In one embodiment of the invention, intact cells and nuclei may be removed by consecutive centrifugation until membranes are substantially free of nuclei and cells, as determined microscopically. For example, lysed cells may be centrifuged at low speed, such as for example, at about 500-2,000 g for about five minutes. The separation procedure is such that less than about 100 cells or nuclei remain in about 2×10⁸ cell equivalents (c.e.) of membrane material. The retrieved supernatant contains membranes which, for example, may be pelleted by ultracentrifugation at about 100,000 g for about 90 minutes. The pellet contains mainly membranes. Membranes may be resuspended, for example, in about 8% sucrose, 5 mM Tris, pH 7.6 and frozen at about −80° C. until use. Any diluent may be used, preferably one that acts as a stabilizer. To determine the quality of membrane preparation, a fraction (about 6×10⁷ c.e. membranes) may be cultured regularly. Cell colonies should not develop and cells or nuclei should not be detected by light microscopy.

Modification of the prepared cells or membranes with DNP or another hapten may be performed by known methods, e.g. by the method of Miller and Claman (J. Immunol., 1976;117:1519) which involves a 30 minute incubation of tumor cells or membranes with a hapten under sterile conditions, followed by washing with sterile saline. Hapten-modification may be confirmed by flow cytometry using a monoclonal anti-hapten antibody.

The dissociated cells or isolated membranes may be used fresh or stored frozen, such as in a controlled rate freezer or in liquid nitrogen until needed. The cells and membranes are ready for use upon thawing. Preferably, the cells or membranes are thawed shortly before they are to be administered to a subject. For example, the cells or membranes may be thawed on the day that a subject is to be skin tested or treated.

Allogeneic tumor cell membranes may be prepared as described above. However, prior to administration to a subject the preparation may be co-incubated with syngeneic (e.g. autologous) antigen presenting cells. Syngeneic antigen-presenting cells process allogeneic membranes such that the patient's cell-mediated immune system may respond to them. This approach permits immunization of a patient with tumor cell membranes originating from a source other than the patient's own tumor. Allogeneic tumor cell membranes are incubated with antigen-presenting cells for a time period varying from about a couple of hours to about several days. The membrane-pulsed antigen presenting cells are then washed and injected into the patient.

Antigen-presenting cells may be prepared in a number of ways including for example the methods of Grabbe et al. (Immunol. Today, 1995;16:117-121) and Siena et al. (Exp. Hematol., 1995;23:1463-1471). Briefly, blood is obtained, for example by venipuncture, from the patient to be immunized. Alternatively, a sample of bone marrow may be collected. Alternatively, blood leukocytes may be obtained by leukapheresis. From any of these sources, mononuclear leukocytes are isolated by gradient centrifugation. The leukocytes may be further purified by positive selection with a monoclonal antibody to the antigen, CD34. The purified leukocytes are cultured and expanded in tissue culture medium (for example, RPMI-1640 supplemented with serum, such as fetal calf serum, pooled human serum, or autologous serum). Alternatively, serum-free medium may be used. To stimulate the growth of antigen-presenting cells, cytokines may be added to the culture medium. Cytokines include but are not limited to granulocyte macrophage-colony stimulating factor (GM-CSF), interleukin 4 (IL4), TNF (tumor necrosis factor), interleukin 3 (IL3), FLT3 ligand and granulocyte colony stimulating factor (G-CSF).

The antigen-presenting cells isolated and expanded in culture, for example, may be characterized as dendritic cells, monocytes, macrophages, and Langerhans cells.

### Tumor Cell Peptides

The isolation of peptides to be used in hapten-modified anti-cancer vaccines is described in U.S. patent application Ser. No. 08/479,016, filed Jun. 7, 1995 and patent application Ser. No. 09/447,897, filed Nov. 24, 1998. Both applications disclose extraction and isolation of hapten-modified peptides, which can be adapted for the present invention. Peptides can also be synthesized based on known sequences, or isolated prior to haptenization. The isolated peptides can then be modified by dual-haptenization.

For purposes of the present invention, peptides are compounds of two or more amino acids and include proteins. Peptides will preferably be of low molecular weight, of about 1,000 kD to about 10,000 kD, more preferably about 1,000 kD to about 5,000 kD, which are isolated from a haptenized tumor cell and which stimulate T cell lymphocytes to produce gamma interferon. The peptide of the invention may be from about 8 to about 20 amino acids, preferably from about 8 to about 12 amino acids. In addition, the peptide is preferably haptenized. Peptides may be isolated from the cell surface, cell interior, or any combination of the two locations. The extract may be particular to type of cancer cell (versus normal cell). The peptides of the present invention include but are not limited to peptides which bind to MHC molecules, a cell surface-associated protein, a peptide associated with a heat shock protein/chaperonin, a protein encoded by cancer oncogenes, or mutated anti-oncogenes. In one preferred embodiment of the invention, peptides are bound to the MHC molecules. For purposes of the present invention “a peptide equivalent” is the peptide having the same amino acid sequence as the peptide isolated from an MHC molecule, although prepared either by degradation of a protein comprising the peptide, synthesized in vitro or recombinant DNA technology.

Preferably, the peptides are derived from tumor specific antigens. There is substantial evidence that the same T-cell-defined tumor antigens are expressed by different human melanoma tumors, suggesting that transformation-associated events may give rise to recurrent expression of the same tumor antigen in tumors of related tissue and/or cellular origin (Sahasrabudhe et al., J. Immunol., 1993; 151:6302-6310; Shamamian et al., Cancer Immunol. Immunother., 1994;39:73-83; Cox et al., Science, 1994;264:716; Peoples et al., J. Immunol., 1993;151:5481-5491; Jerome et al., Cancer Res., 1991;51:2908-2916; Morioke et al., J. Immunol., 1994;153:5650-5658). Examples of such antigens from human tumors include, but are not limited to, MART 1/Melan A, gp-100, and tyrosinase (melanoma); MAGE-1 and MAGE-3 (bladder, head and neck, non-small cell carcinoma); HPV E6 and E7 proteins (cervical cancer); HER2/neu/c-erbB-2 (breast cancer); HER3, HER4, Mucin (MUC-1) (breast, pancreas, colon, prostate); prostate specific antigen (PSA) (prostate); and CEA (colon, breast, GI).

The cell extracts of the invention, including peptides originally isolated from MHC molecules located on tumor cell plasma membranes, have the property of stimulating T cells. For purposes of the present invention, stimulation refers to proliferation of T cells as well as production of cytokines by T cells in response to the cell extract. Proliferation of T cells may be observed by uptake by T cells of modified nucleic acids, such as but not limited to ³H thymidine, ¹²⁵IUDR (iododeoxyuridine); and dyes such as 3-(4,5-dimethylthiazol-2-yl)-2,5-diphenyltetrazolium bromide (MTT) which stains intact cells. In addition, production of cytokines such as but not limited to γ-interferon (INFγ), tumor necrosis factor (TNF), and interleukin-2 (IL-2) may be tested. Production of cytokines is preferably in an amount greater than 15 picograms/ml, more preferably about 20 to about 30 picograms/ml, even more preferably about 50 picograms/ml. Alternatively, cytotoxicity assays can be used to evaluate T cell stimulation.

From the hapten-modified cells, peptides may be extracted, some of which are hapten-modified as a result of modifying the cells. Alternatively, extracted or synthetic peptides can be reacted with a hapten after isolation or synthesis. Protein extraction techniques known to those of skill in the art may be followed by antigen assays to isolate proteins or peptides effective for patient treatment. The methods of isolating cell extracts are readily known to those skilled in the art. Briefly, cancer cells are isolated from a tumor and cultured in vitro. A hapten preparation is added to the cultured cells in accordance with the method set forth above. Peptides are isolated from cells according to an established technique, e.g., the technique of Rotzschke et al. (Nature, 1990;348:252), the disclosure of which is hereby incorporated by reference in its entirety. The cells are treated with a weak acid such as but not limited to trifluoroacetic acid (TFA). The cells are thereafter centrifuged and the supernatant is saved. Compounds having a molecular weight greater than 5,000 are removed from the supernatant by gel filtration (G25 Sepharose, Pharmacia). The remainder of the supernatant is separated on a reversed-phase HPLC column (Superpac Pep S, Pharmacia LKB) in 0.1% TFA using a gradient of increasing acetonitrile concentration; flow rate=1 ml/min, fraction size=1 ml. Fractions containing small peptides are collected by HPLC according to the method of Sambrook et al., Molecular Cloning: A Laboratory Manual, 2nd ed., Cold Spring Harbor Laboratory Press, Cold Spring Harbor, N.Y. (1989), concentrated, and frozen.

The HPLC fractions containing small peptides are screened for immunological activity, e.g., by allowing them to bind to autologous B lymphoblastoid cells which are then tested for their ability to stimulate tumor-specific T lymphocytes. T cells used for this testing are isolated from a human patient and propagated in vitro as described in PCT Publication No. WO98/14206. The peptides that stimulate T cells are then analyzed for their structure. For example, the peptides are sequenced using methods known in the art to determine their amino acid sequence. In one embodiment of the invention, the peptides are sequenced as a pool as described by Burrows et al. (J. NeuroSci. Res., 1997;49:107-116) and Gavin et al. (Eur. J. Immunol., 1994;24:2124-33) to determine prevailing motifs. In another embodiment of the invention, the peptides are further separated using methods known in the art, such as HPLC, as described in U.S. Pat. Nos. 5,747,269; 5,487,982; 5,827,516 and 5,820,862 and sequenced. Sequencing is performed by using Edman degradation as described in Edman and Berg, Eur. J. Biochem., 1967;80:116-132, or any modification thereof known in the art. One powerful technique for characterizing isolated peptides is mass spectrometry.

Once the sequence of the peptides isolated from the MHC molecules is known, synthetic peptides having the same sequence are synthesized and used as a vaccine alone, presented on an antigen presenting cell and/or in combination with other extracts or whole cells using the methods described above. The equivalent peptides may also be produced recombinantly or by chemical degradation of proteins containing the isolated peptides.

In another embodiment, the structure of known peptides is altered by changing at least one amino acid and the so altered peptides are tested for their ability to stimulate T cells.

### Haptenization

The tumor cells, membranes, or peptides can be haptenized. For purposes of the present invention, virtually any small molecule, including peptides, that can induce an immune response when conjugated to a carrier, may function as a hapten. A variety of haptens of different chemical structure have been shown to induce similar types of immune responses: e.g., dinitrophenyl (DNP); trinitrophenyl (TNP) (Kempkes et al., J. Immunol., 1991;147:2467); phosphorylcholine (Jang et al., Eur. J. Immunol., 1991;21:1303); nickel (Pistoor et al., J. Invest. Dermatol., 1995;105:92); and arsenate (Nalefski and Rao, J. Immunol., 1993;150:3806). Conjugation of a hapten to a cell may preferably be accomplished by conjugation via ε-amino groups of lysine or —COOH groups. This group of haptens include a number of chemically diverse compounds: halonitrobenzenes (including dinitrofluorobenzene, difluorodinitrobenzene, trinitrofluorobenzene), N-iodoacetyl-N′-(5-sulfonic-1-naphthyl) ethylene diamine, nitrobenzene sulfonic acids (including trinitrobenzenesulfonic acid and dinitrobenzene sulfonic acid), fluorescein isothiocyanate, arsenic acid benzene isothiocyanate, and dinitrobenzene-S-mustard (Nahas and Leskowitz, Cellular Immunol., 1980;54:241). Once familiar with the present disclosure, skilled artisans would be able to choose haptens for use in the present invention.

Haptens generally include a reactive group for conjugation to a substituent on an amino acid side chain of a protein or polypeptide (e.g., a free carboxylic acid group as in the case of aspartic acid or glutamic acid; the ε-amino group of lysine; the thiol moiety of cysteine; the hydroxyl group of serine or tyrosine; the imidazole moiety of histidine; or the aryl groups of tryptophan, tyrosine, or phenylalanine). As used herein, the term “reactive group” refers to a functional group on the hapten that reacts with a functional group on a peptide or protein. The term “functional group” retains its standard meaning in organic chemistry. These reactive groups on a hapten are termed herein the “hapten reactive group”. Numerous hapten reactive groups are known, which interact with the substituents present on the side chains of amino acids that comprise peptides and proteins. Preferred examples of such reactive groups for conjugation to specific polypeptide substituents are carboxylic acid or sulfonic acid derivatives (including acid chlorides, anhydrides, and reactive carboxylic esters such as N-hydroxysuccinimide esters), imidoesters, diazonium salts, isocyanates, isothiocyanates, halonitrobenzenes, α-halocarbonyl compounds, maleimides, sulfur mustards, nitrogen mustards, and aziridines.

Functional Groups Reactive With Primary Amines.

Hapten reactive groups that would form a covalent bond with primary amines present on amino acid side chains would include, but not be limited to, acid chlorides, anhydrides, reactive esters, α,β-unsaturated ketones, imidoesters, and halonitrobenzenes. Various reactive esters with the capability of reacting with nucleophilic groups such as primary amines are available commercially, e.g., from Pierce (Rockford, Ill.).

Functional Groups Reactive With Carboxylic Acids.

Carboxylic acids in the presence of carbodiimides, such as EDC, can be activated, allowing for interaction with various nucleophiles, including primary and secondary amines. Alkylation of carboxylic acids to form stable esters can be achieved by interaction with sulfur or nitrogen mustards, or haptens containing either an alkyl or aryl aziridine moiety.

Functional Groups Reactive With Aromatic Groups.

Interaction of the aromatic moieties associated with certain amino acids can be accomplished by photoactivation of aryl diazonium compound in the presence of the protein or peptide. Thus, modification of the aryl side chains of histidine, tryptophan, tyrosine, and phenylalanine, particularly histidine and tryptophan, can be achieved by the use of such a reactive functionality.

Functional Groups Reactive With Sulfhydryl Groups.

There are several reactive groups that can be coupled to sulfhydryl groups present on the side chains of amino acids. Haptens containing an α,β-unsaturated ketone or ester moiety, such as maleimide, provide a reactive functionality that can interact with sulfhydryl as well as amino groups. In addition, a reactive disulfde group, such as 2-pyridyldithio group or a 5,5′-dithio-bis-(2-nitrobenzoic acid) group is also applicable. Some examples of reagents containing reactive disulfide bonds include N-succinimidyl 3-(2-pyridyl-dithio) propionate (Carlsson, et al., Biochem J., 1978;173:723-737), sodium S-4-succinimidyloxycarbonyl-alpha-methylbenzylthiosulfate, and 4-succinimidyloxycarbonyl-alpha-methyl-(2-pyridyldithio)toluene. Some examples of reagents comprising reactive groups having a double bond that reacts with a thiol group include succinimidyl 4-(N-maleimidomethyl)cyclohexahe-1-carboxylate and succinimidyl mmaleimidobenzoate.

Other functional molecules include succinimidyl 3-(maleimido)propionate, sulfosuccinimidyl 4-(p-maleimido-phenyl)butyrate, sulfosuccinimidyl 4-(N-maleimidomethylcyclohexane)-1-carboxylate, maleimidobenzoyl-N-hydroxy-succinimide ester. Many of the above-mentioned reagents and their sulfonate salts are available from Pierce.

Haptens also include a hapten recognition group that interacts with antibody. The recognition group is irreversibly associated with the hapten reactive group. Thus, when the hapten reactive group is conjugated to a functional group on the target molecule, the hapten recognition group is available for binding with antibody. By selecting an appropriate hapten reactive group, antibody recognition of, and binding to, a hapten recognition group can be made independent of the functional group to which the hapten is conjugated. When this is the case, the haptens are functionally equivalent, and are said to share antibody binding features. Naturally, in cases where the recognition groups of two haptens differ chemically, the reactive groups may be the same or different, i.e., reactive with the same or different functional groups on the target molecule.

Examples of different hapten recognition groups include without limitation to dinitiophenyl, trinitrophenyl, fluorescein, other aromatics, phosphorylcholine, peptides, advanced glycosylation endproducts (AGE), carbohydrates, etc.

In a specific embodiment, the same hapten recognition group can be coupled to different amino acids through different hapten reactive groups. For example, the reagents dinitrobenzene sulfonic acid, dinitro-phenyldiazonium, and dinitrobenzene-S-mustard, all form the dinitrophenyl hapten coupled to amino groups, aromatic groups, and carboxylic acid groups, respectively. Similarly, an arsonic acid hapten can be coupled by reacting arsonic acid benzene isothiocyanate to amino groups or azobenzenearsonate to aromatic groups.

### Isolation and Haptenization of Tumor Cells

The dissociated cells, cell membranes, or peptides may be stored frozen in a freezing medium (e.g., prepared from a sterile-filtered solution of 50 ml of serum albumin added to 450 ml of RPMI 1640 (Mediatech) supplemented with L-glutamine and adjusted to an appropriate pH with NaOH), such as in a controlled rate freezer or in liquid nitrogen until needed. The cells are ready for use upon thawing. Preferably, the cells are thawed shortly before hapenization. Optionally, the cells may be washed, and optionally irradiated to receive a dose of about 2500 cGy. They may then be washed again and suspended in Hanks Balanced Salt Solution (HBSS) without phenol red and without HSA.

Modification of the prepared cells with DNP or another hapten may be performed by known methods, e.g. by the method of Miller and Clanian (J. Immunol., 1976;117:151), incorporated herein by reference in its entirety, which involves a 30 minute incubation of tumor cells with DNFB under sterile conditions, followed by washing with sterile saline or HBSS/HSA.

### Vaccine Preparations

The compositions of the invention may be administered in a mixture or in combination with a pharmaceutically-acceptable carrier, selected with regard to the intended route of administration and standard pharmaceutical practice. Dosages may be set with regards to weight, and the clinical condition of the patient. The proportional ratio of active ingredient to carrier naturally depend on the chemical nature, solubility, and stability of the compositions, as well as the dosage contemplated. The amount of the tumor cells of the invention to be used depend on such factors as the affinity of the compound for cancer cells, the amount of cancer cells present, and the solubility of the composition. The compounds of the present invention may be administered by any suitable route, including inoculation and injection via, for example, intradermal, intravenous, intraperitoneal, intramuscular, and subcutaneous routes.

In a preferred embodiment of the invention, the composition to be used for the first “priming” dose comprises a vaccine comprising about 2×10⁵ to about 2.5×10⁶, more preferably less than about 2×10⁶, even more preferably less than about 1×10⁶, growth-incapacitated tumor cells or tumor cell equivalents suspended in a pharmaceutically acceptable carrier or diluent, such as but not limited to Hanks solution, saline, phosphate-buffered saline (PBS), and water. The composition may be administered by intradermal injection into 3 contiguous sites per administration on the legs, preferably excluding limbs ipsilateral to a lymph node dissection. Vaccine preparations for subsequent administrations can comprise from about 2×10⁵ to about 1×10⁷ tumor cells or tumor cell equivalents, preferably from about 1×10⁶ to about 2.5×10⁶ tumor cells or tumor cell equivalents.

### Formulations

The formulations according to the invention may be prepared in various ways. The different components may be mixed together, and then added to haptenized tumor cells or tumor cell equivalents. It is also possible to mix one or several of the components with the haptenized tumor cell preparation and then add the remaining component(s). The preparation of the formulation and its addition of the haptenized tumor cells are preferably performed under sterile conditions.

The respective proportions of the components of the media according to the invention may be adapted by persons skilled in the art.

Generally, serum albumin, preferably autologous to the species of animal in which the tumor cell preparation will be administered, will be added to an appropriate buffered cell culture medium. For example, “human serum albumin” or “HSA” refers to a non-glycosylated monomeric protein consisting of 585 amino acid residues, having a molecular weight of about 66 kD. Its globular structure is maintained by 17 disulfide bridges, which create a sequential series of 9 double loops (Brown, “Albumin structure, function and uses”, Rosenoer, V. M. et al. (eds.), Pergamon Press:Oxford, pp. 27-51, 1977). The genes encoding for HSA are known to be highly polymorphic, and more than 30 apparently different genetic variants have been identified by electrophoretic analysis (Weitkamp, L. R. et al., Ann. Hum. Genet., 1973;37:219-226). The HSA gene comprises 15 exons and 14 introns corresponding to 16,961 nucleotides from the putative mRNA “capping” site up to the first site of addition of poly(A).

In its essence, a buffered cell culture medium is an isotonic buffered aqueous solution, such as phosphate buffered saline, Tris-buffered saline, or HEPES buffered saline. In a preferred embodiment, the medium is plain Hank's medium (no phenol red), e.g., as sold commercially by Sigma Chemical Co. (St. Louis, Mo., USA). Other tissue culture media can also be used, including basal medium Eagle (with either Earle's or Hank's salts), Dulbecco's modified, Eagle's medium (DMEM), Iscove's modified Dulbecco's medium (IMDM), Medium 199, Minimal Essential Medium (MEM) Eagle (with Earle's or Hank's salts), RPMI, Dulbecco's phosphate buffered salts, Earle's balanced salts (EBSS), and Hank's Balanced Salts (HBSS). These media can be supplemented, e.g., with glucose, Ham's nutrients, or HEPES. Other components, such as sodium bicarbonate and L-glutamine, can be specifically included or omitted. Media, salts, and other reagents can be purchased from numerous sources, including Sigma, Gibco, BRL, Mediatech, and other companies. For use in humans, an appropriate medium is pharmaceutically acceptable.

Preferably, a formulation of whole, intact cells comprises an optimized serum albumin concentration in a buffered cultured medium, preferably HBSS. In a specific embodiment, the final concentration of serum albumin is about 1.0% in a HBSS. However, an unexpected improvement in cell viability can be achieved using at least about 0.25% serum albumin, a greater improvement in cell viability with 0.3% serum albumin (as compared to 0.11% serum albumin), and an even greater improvement is possible using at least about 0.5% serum albumin. Upper limits to the concentration are determined by the need to avoid contaminants that may be present in naturally-derived serum albumin, or alternatively to avoid allergic reactions to recombinant serum albumin. Preferably, the concentration of serum albumin in a formulation of the invention is no more than about 10%. More preferably, the concentration is less than or equal to about 5% and, more preferably still, less than or equal to about 2%.

Also, a composition or formulation of the invention may contain other components in addition to serum albumin to further stabilize the haptenized tumor cells. Examples of such components include, but are not limited to, carbohydrates and sugars, such as dextrose, sucrose, glucose, and the like, e.g., at a 5% concentration; medium to long chain polyols, such as glycerol, polyethylene glycol, and the like, e.g., at 10% concentration; other proteins; amino acids; nucleic acids; chelators; proteolysis inhibitors; preservatives; and other components. Preferably, any such constituent of a composition of the invention is pharmaceutically acceptable.

### Adjuvant

In preferred embodiment, the tumor cell vaccines administered subsequent to the “priming” dose may be administered with an immunological adjuvant. The term “adjuvant” refers to a compound or mixture that enhances the immune response to an antigen (Hood et al., Immunology, Second Edition, 1984, Benjamin-Cummings: Menlo Park, Calif., p. 384). While commercially available pharmaceutically acceptable adjuvants are limited, representative examples of adjuvants include Bacille Calmette-Guerin (BCG) the synthetic adjuvant QS-21 comprising a homogeneous saponin purified from the bark of Quillaja saponaria and Corynebacterium parvum (McCune et al., Cancer, 1979;43:1619). Other adjuvants include Complete and Incomplete Freund's Adjuvant, detoxified endotoxins, mineral oils, surface active substances such as lipolecithin, pluronic polyols, polyanions, peptides, and oil or hydrocarbon emulsions. In some cases, immunostimulatory compounds, as exemplified below, may function as adjuvants.

It will be understood that the adjuvant is subject to optimization. In other words, the skilled artisan can engage in experimentation that is no more than routine to determine the best adjuvant to use.

### Immunomodulators

Treatment regimens for haptenized tumor cells or tumor cell extracts may include immunodulators, i.e. drugs that alter, suppress or strengthen the body's immune system. Immunopotentiators herein means compounds that potentiate the immune system for treatment with a tumor vaccine according to the present invention. Preferably, when administered in a treatment regimen based upon haptenized tumor cells or cell extracts, immunopotentiators at least temporarily diminish any down-regulation of anti-tumor responses evoked by the tumor vaccine, while to a lesser extent affecting other parts of the immune system. One preferred example of such a compound include, but is not limited to a low dose, cyclophosphamide. Immunosuppressants include chemotherapeutic agents, and may administered alone, or co-administered with the haptenized tumor cell vaccine, as appropriate. Immunostimulators include, but are not limited to cytokines such as interferon (IFN)-γ, IL-2, IL-6, IL-12, IL-13, GM-CSF, G-CSF and pro-inflammatory mediators such as endotoxin, TNF, IL-1, etc. In another embodiment, a CTLA-4 receptor antagonist, such as an anti-CTLA-4 antibody, can be used to prevent signaling for termination of T cell activation.

### Combination Therapies

The haptenized tumor cell compositions may be co-administered with other compounds including but not limited to an immunostimulatory agent or agents. The tumor cells and extracts of the invention may also be used in conjunction with other cancer treatments including but not limited to chemotherapy, radiation therapy, immunotherapy, and gene therapy.

Clinical Response Criteria

The following standard criteria may be used: Complete response indicates complete disappearance of all metastases for at least about one month, more preferably for at least about three months, without development of new metastases; Partial response indicates at least about 50% reduction in the mean diameter of a measurable metastasis for at least about one month, more preferably for at least about three months, without development of new metastases; Mixed response indicates at least about 50% reduction in the mean diameter of a measurable metastasis with concomitant growth of another metastasis; Stable disease indicates more than about 25% increase in the mean diameter of any measurable metastasis. Other clinical response criteria that may be used include prolongation of survival and prolongation of time to relapse (relapse-free survival).

### EXAMPLES

The following examples are illustrative of the invention, but not limiting thereof.

Example 1

Immunotherapy of Spontaneous Murine Carcinoma Tumor

The immunotherapeutic protocol employed in our studies was similar to the protocol employed by Berd et al as a postsurgical adjuvant therapy for melanoma patients with clinically evident nodal metastases (Berd et al., J Clin Oncol 1997;15:2359). Specifically, after surgical excision of the primary 410.4 mammary tumor, the mice were given low-dose CY followed by vaccination with DNP-modified, γ-irradiated, autologous tumor cells admixed with BCG. Vaccination with DNP-modified, γ-irradiated, autologous tumor cells admixed with BCG offered therapeutic benefits against established metastases also in the 410.4 tumor model.

### Materials and Methods

Tumor Cells.

The highly metastatic 410.4 tumor (Miller, Invasion Metastasis 1981;1:220; Pulaski et al., Cancer Res 1998;58:1486; Miller, Invasion Metastasis 1983;3:234) that originated from a spontaneously arising murine mammary carcinoma (Heppner et al., Cancer Res 1978;38:3758; Miller et al., Cancer Res 1981;41:3863) was used throughout our studies. Tumor cells were maintained in vitro at 37° C. in 5% CO₂ in Falcon 75 cm² polystyrene tissue culture flasks (Becton Dickinson Labware, Franklin lakes, N.J.) in Dulbecco's modified Eagle medium (DMEM) supplemented with 10% fetal bovine serum and 100 units/ml penicillin and 100 mg/ml streptomycin. Every 2-3 days the tumor cells were detached with trypsin-EDTA solution (Life Technologies Inc., Grand island, N.Y.) and 2.0×10⁶ cells in 20 ml of medium were seeded per new flask.

In vivo Tumor Model.

In vitro grown 410.4 tumor cells were detached with trypsin-EDTA, and 3×10⁵ 410.4 tumor cells in 0.2 ml RPMI-1640 medium (Life Technologies Inc.) were injected into the mammary fatpads of female BALB/cAnNCrlBR mice 7 to 10 weeks old (Charles Rivers Breeding Laboratories, Wilmington, Mass.). When the tumors reached 6-8 mm in diameter, the tumors were surgically excised. Unless otherwise stated, five to eight days after tumor excision, the mice were divided into groups and subjected to our experimental design.

Vaccine Preparation.

On the day of vaccination, in vitro grown 410.4 tumor cells were detached with 0.02% EDTA solution (without trypsin) (Sigma Chemical Co., St Louis, MO) followed by forceful pipetting, and the tumor cells were then subjected to γ-irradiation (2500 cGY from a Cs-137 source; The J. L. Sepherd and Associates, Model 143-68 irradiator). Subsequently, an aliquot of the γ-irradiated 410.4 cells was DNP modified by exposure to dinitrofluorobenzene (DNFB, Sigma Chemicals Co., St. Louis, Mo.), according to the protocol of Berd et al (J Clin Oncol 1997;15:2359). Each vaccine was administered in a total volume of 0.2 ml and consisted of 3-5×10⁶ unmodified or DNP-modified, γ-irradiated, tumor cells admixed with 0.5 to 4×10⁶ colony-forming units (CFU) of bacille Calmette-Guerin (BCG, Tice strain).

Study Design.

Unless otherwise stated, on day five to eight after tumor excision mice received an i.p. injection of 15 mg/kg cyclophosphamide (CY; Mead Johnson—A Bristol-Myers Squibb Co., Princeton, N.J.). Three days after the low-dose CY treatment, the mice received a s.c. injection of unmodified, or DNP-modified, γ-irradiated, autologous tumor cell vaccine close to the site of tumor excision. This protocol was repeated every 10 days for the duration of the experiment. The mice were monitored twice a week for the appearance of visible metastases and the results are presented as percentage of relapse-free survival among all mice subjected to the same treatment protocol.

Statistical Analysis.

The percentages of relapse-free survival of mice subjected to different treatment protocols were compared at various time points after tumor excision by the use of paired Student's t test. A p value of 0.05 or lower was considered significant.

### Results

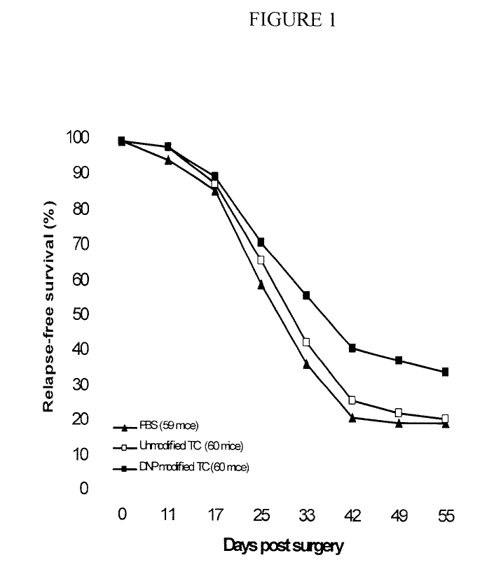

Experiments were undertaken to assess the therapeutic benefits offered by low-dose CY followed by DNP-modified, γ-irradiated, autologous tumor cell vaccine against metastatic disease in mice in which the primary tumor was surgically excised when it reached 6-8 mm in diameter. Mice were monitored for the appearance of visible metastases every 3-4 days and the percentage of relapse-free survival among all mice subjected to this therapeutic protocol was compared at various time points after tumor excision to the percentage of relapse-free survival among a second group of the same batch of mice from which the primary tumor was surgically removed and which were subjected to the same therapeutic regimen with unmodified (instead of with DNP-modified), γ-irradiated, autologous tumor cell vaccine. As a reference point, we provide information about the percentage of relapse-free survival in a third group of the same batch of mice from which the primary tumor was surgically excised and which received only PBS. FIG. 1 depicts the cumulative data from 5 experiments with 59-60 mice per group.

The relapse-free survival of mice that received DNP-modified, γ-irradiated, autologous tumor cell vaccine was significantly better than the relapse-free survival of mice that received unmodified, γ-irradiated, autologous tumor cell vaccine (p=0.01), or mice that received PBS (p=0.003). Thus, similar to the observations of Berd et al (J Clin Oncol 1997;15:2359) in patients with metastatic melanoma, DNP-modified, γ-irradiated, autologous tumor cell vaccine offers therapeutic benefits against metastatic disease also in the murine 410.4 tumor model.

### Example 2

CD8⁺ Cells are Essential for an Effective Anti-tumor Response

This Example provides data illustrating that CD8⁺ T-cells are actually essential for the realization of the therapeutic benefits of DNP-modified, γ-irradiated, autologous tumor cell vaccine (preceded by low-dose CY) against metastatic disease, by demonstrating that in vivo depletion of CD8⁺ T-cells abrogated the therapeutic benefits offered by this immunotherapeutic protocol in the 410.4 tumor model.

### Materials and Methods

All “Materials and Methods” are the same as for Example 1, with the following modification.

In vivo depletion of CD8⁺ T-cells. The in vivo depletion of CD8⁺ T-cells was done as previously described (Takesue et al., Cancer Res 1990;50:7641; La Motte et al., J Immunol 1999;162:4817). Briefly, mice were given an i.p. injection of 1 mg/mouse of anti-CD8 monoclonal antibody (mAb) 2.43, starting 1 day before the first injection of unmodified or DNP-modified, γ-irradiated, autologous tumor cell vaccine and repeated every 7 days thereafter for the duration of the experiment. This protocol of anti-CD8 mAb treatments was found by indirect immunofluorescence staining followed by flow-cytometric analysis to lead to >90% depletion of CD8⁺ cells from the lymph nodes of the treated mice, with no decrease, but actually some increase, in the percentage of CD4⁺ T-cells.

### Results

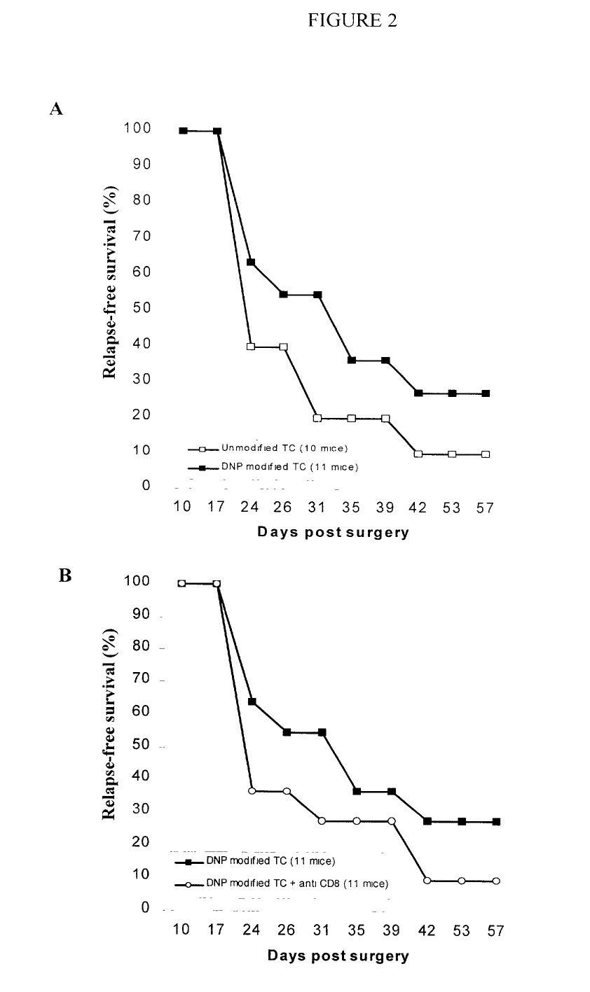

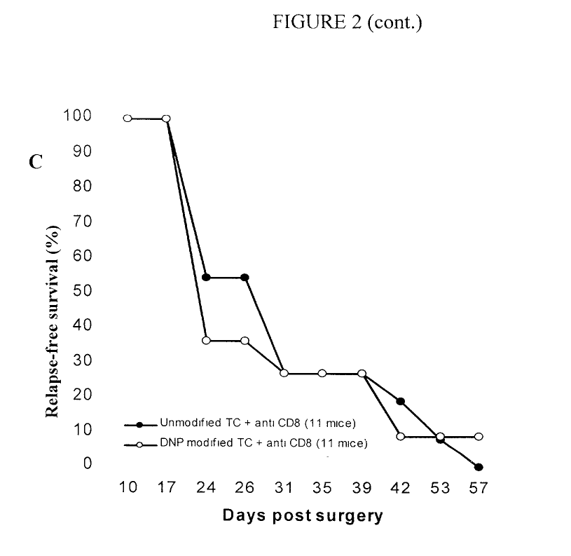

In light of reports by Berd et al suggesting that CD8⁺ T-cells may be important for the anti-metastatic effects of DNP-modified, γ-irradiated, autologous tumor cell vaccine in patients with metastatic melanoma Berd et al., Cancer Res 1991;51:2731; Sato, Cancer Immunol Immunotherapy 1996;43:174; Sensi et al, J Clin Invest 1997;99:710), experiments were undertaken to assess directly the importance of CD8⁺ T-cells for the anti-metastatic effects of DNP-modified, γ-irradiated, autologous tumor cell vaccine in the 410.4 tumor model. Specifically, we assessed the effect of in vivo depletion of CD8⁺ T-cells on the therapeutic benefits offered by DNP-modified, γ-irradiated, autologous tumor cell vaccine (preceded by low-dose CY) against metastatic disease in the murine 410.4 tumor model. As seen in FIG. 2a, and in confirmation of the results presented in FIG. 1, treatment of mice (from which the primary tumor was surgically excised) with DNP-modified, γ-irradiated, autologous tumor cell vaccine led to a significantly better relapse-free survival than treatment of such mice with unmodified, γ-irradiated, autologous tumor cell vaccine (p=0.001). However, the relapse-free survival of mice treated with DNP-modified, γ-irradiated, autologous tumor cell vaccine was significantly worse when the mice were depleted of CD8⁺ T-cells (FIG. 2b; p=0.001). In fact, the relapse-free survival of mice depleted of CD8⁺ T-cells and treated with DNP-modified, γ-irradiated, autologous tumor cell vaccine, was not significantly different than that of mice depleted of CD8⁺ T-cells and treated with unmodified, γ-irradiated, autologous tumor cell vaccine (FIG. 2C; p=0.25). Thus, CD8⁺ T-cells are important for the realization of the effectiveness of DNP-modified, γ-irradiated, autologous tumor cell vaccine (preceded by low-dose CY) against metastatic disease.

### Example 3

Pretreatment With DNP-modified Autologous Tumor Cells

Given the fact that the anti-metastatic effects induced by DNP-modified, γ-irradiated, autologous tumor cell vaccine (preceded by low-dose CY) in the 410.4 tumor model resembles the anti-metastatic effects induced by this regimen in patients with metastatic melanoma, we initiated studies to determine if a modified protocol can offer even greater therapeutic benefits. Towards this goal, we determined if pretreatment of mice with a single dose of DNP-modified, γ-irradiated, autologous tumor cells (without BCG) prior to initiation of the low-dose CY treatment followed by DNP-modified, γ-irradiated, autologous tumor cell vaccine would be superior in terms of anti-metastatic activity to low-dose CY followed by DNP-modified, γ-irradiated, autologous tumor cell vaccine without the pretreatment regimen. The results provided herein illustrate that pretreatment of mice with the DNP-modified, γ-irradiated, autologous tumor cells (without BCG) prior to initiation of the low-dose CY treatment followed by DNP-modified, γ-irradiated, autologous tumor cell vaccine offers further therapeutic benefits in the 410.4 tumor model. These data suggest that pretreatment with DNP-modified, γ-irradiated, autologous tumor cells prior to initiation of the low-dose CY treatment followed by DNP-modified, γ-irradiated, autologous tumor cell vaccine may offer further therapeutic benefits also in patients with metastatic melanoma.

### Materials and Methods

All “Materials and Methods” are the same as for Example 1, with the following modification.

Pretreatment Protocol.

The pretreatment protocol consisted of a single s.c. injection of 1×10⁶ DNP-modified, γ-irradiated, autologous tumor cell (without BCG) administered on the back of the mice on day 3-7 after the excision of the primary tumor. Three to seven days later, the mice received low-dose CY followed by DNP-modified, γ-irradiated, autologous tumor cell vaccine.

### Results

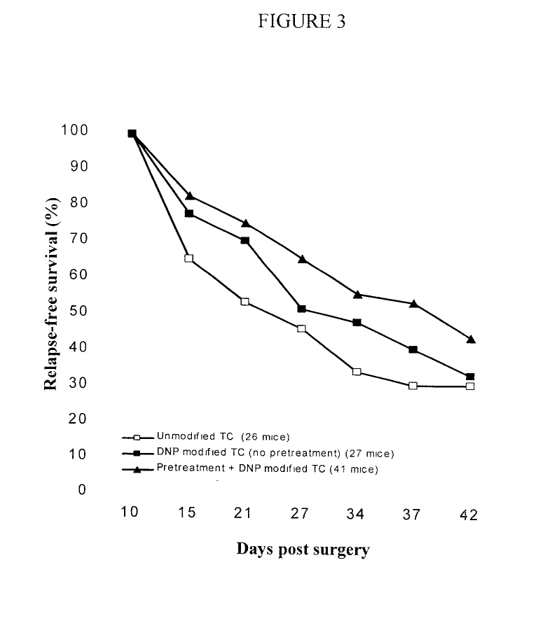

Experiments were undertaken to determine if pretreatment of mice with a single dose of DNP-modified, γ-irradiated, autologous tumor cells (without BCG) prior to initiation of the low-dose CY treatment followed by DNP-modified, γ-irradiated, autologous tumor cell vaccine would be superior in terms of anti-metastatic activity to low-dose CY followed by DNP-modified, γ-irradiated, autologous tumor cell vaccine without the pretreatment regimen. As seen in FIG. 3, and in confirmation of the results presented in FIGS. 1 and 2a, treatment of mice (from which the primary tumor was surgically excised) with low-dose CY followed by DNP-modified, γ-irradiated, autologous tumor cell vaccine led to a significantly better relapse-free survival than treatment of such mice with low-dose CY followed by unmodified, γ-irradiated, autologous tumor cell vaccine (p=0.005). Interestingly, pretreatment of mice with DNP-modified, γ-irradiated, autologous tumor cells (without BCG) 3-7 days prior to the initiation of the low-dose CY treatment followed by DNP-modified, γ-irradiated, autologous tumor cell vaccine resulted in a significant improvement in the relapse-free survival relative to that observed in mice that received low-dose CY followed by DNP-modified, γ-irradiated, autologous tumor cell vaccine without the pretreatment regimen (p=0.002). Thus, pretreatment of mice with a single dose of DNP-modified, γ-irradiated, autologous tumor cells (without BCG) prior to the initiation of the low-dose CY treatment followed by DNP-modified, γ-irradiated, autologous tumor cell vaccine offers further therapeutic benefits against metastatic disease in the 410.4 tumor model.

### Discussion

These Examples report development of an animal model that can provide information on the effectiveness of postsurgical immunotherapies for recurrence of metastatic disease. Examples 1 and 2 show that treatment of mice from which the primary tumor was excised with DNP-modified, γ-irradiated autologous tumor cell vaccine (preceded by low-dose CY) is effective in reducing the relapse rate of metastases in these animals via a CD8⁺ T-cell-dependent mechanism. Examples 3 shows that pretreatment of mice with DNP-modified, γ-irradiated, autologous tumor cells prior to the initiation of the above regimen enhances the efficacy of our vaccination protocol against metastatic disease.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all numerical values are approximate and are provided for description only.

Patents, patent applications, and publications cited throughout this application are incorporated herein by reference in their entireties.

What is claimed is:

1. A method for testing the efficacy of an immunotherapy of a tumor in a non-human animal, which method comprises evaluating an anti-tumor response to a tumor immunotherapy administered to a non-human animal harboring a tumor.

2. The method according to claim 1, wherein the animal is a mouse.

3. The method according to claim 1, wherein the tumor is a carcinoma.

4. The method according to claim 1, wherein the immunotherapy comprises administering a hapten-modified tumor cell preparation, wherein any tumor cells are rendered incapable of growth or multiplication in the animal.

5. The method according to claim 4, wherein the tumor cell extract is selected from the group consisting of tumor cell membranes and tumor cell polypeptides.

6. The method according to claim 1, wherein the immunotherapy further comprises treating the animal with an immunomodulatory agent that potentiates protective anti-tumor immunity or inhibits immune suppression, or both.

7. The method according to claim 6, wherein the immunomodulatory agent is cyclophosphamide.

8. The method according to claim 6, further comprising administering a tumor cell composition free of any adjuvant to the animal prior to administering the immunomodulatory compound.

9. The method according to claim 4, further comprising comparing the anti-tumor response to an anti-tumor response in a control animal of the same species as the non-human animal treated with a control immunotherapy, wherein the control immunotherapy comprises administering a non-hapten-modified tumor cell preparation, wherein any tumor cells are rendered incapable of growth or multiplication.

10. A method for testing the efficacy of an immunotherapy of a carcinoma tumor in a mouse, which method comprises evaluating an anti-tumor response to a carcinoma immunotherapy administered to a mouse harboring a carcinoma tumor treated with an immunotherapy, wherein the immunotherapy comprises administering a composition comprising a hapten-modified carcinoma tumor cell preparation to the mouse.

11. The method according to claim 10, wherein the mouse is a BALB/c mouse.

12. The method according to claim 11, wherein the carcinoma is a 410.4 mammary carcinoma.

13. The method according to claim 10, which further comprises administering a first (priming) dose of the composition without any adjuvant.

14. The method according to claim 13, wherein the composition is administered prior to administration of an immunomodulatory agent that potentiates protective anti-tumor immunity or inhibits immune suppression, or both.

15. The method according to claim 14, wherein the immunomodulatory agent is cyclophosphamide.

16. The method according to claim 14, wherein a second composition comprising an adjuvant and a hapten-modified carcinoma tumor cell preparation, which contains from about 2×10⁵ to about 1×10⁷ tumor cells or tumor cell equivalents, is administered after the immunomodulatory agent.

17. The method according to claim 16, wherein the adjuvant is selected from the group consisting of Bacille Calmette-Guerin, Q-21, and detoxified endotoxin.
